(19) United States
(12) Patent Application Publication
(10) Pub. No.: US 2026/0220924 A1
(43) Pub. Date: Jul. 30, 2026

(54) CLASSIFYING IMAGE FEATURES OF A CARDIAC IMAGES

(71) Applicant: FEops NV, Gent (BE)

(72) Inventors: Maxime NAUWYNCK, Brugge (BE); Eva HEFFINCK, Kortrijk (BE); Kilian MICHIELS, Oostduinkerke (BE)

(21) Appl. No.: 19/144,916

(22) PCT Filed: Jan. 3, 2024
(86) PCT No.: PCT/EP2024/050066

§ 371 Date: Jun. 30, 2025

(30) Foreign Application Priority Data
Jan. 3, 2023 (EP) 23150196.6

(51) Int. Cl. G06V 10/776; A61B 5/00; G06T 7/00; G06V 10/25; G06V 10/764; G06V 10/774; G06V 10/82; G16H 30/20

(52) U.S. Cl. CPC G06V 10/776; A61B 5/0044; G06T 7/0012; G06V 10/25; G06V 10/764; G06V 10/774; G06V 10/82; G16H 30/20; G06T 2207/10081; G06T 2207/10088; G06T 2207/10104; G06T 2207/10132; G06T 2207/20036; G06T 2207/20076; G06T 2207/20081; G06T 2207/20084; G06T 2207/20104; G06T 2207/30048; G06V 2201/031

(57) ABSTRACT

The invention is directed at a classifying image features (13) to determine a cardiac characteristic, using a data processing model (23) trained to evaluate a probability that the image feature (13) matches a certain class (25). According to the method (50), the data processing model (23) receives cardiac image data (32), and obtains (54) a region of interest. Probability data per class is collected (56) from the data processing model (23) and evaluated (58) to identify candidate classes likely of matching the image feature. Dependent on a certainty level (60, 64) a next step is performed. If there's one candidate class and the certainty exceeds a threshold, the candidate class will be associated with the image feature (68). If the certainty is below threshold user input will be requested (70). If there are more candidate classes, user input will also be requested (72).

### FIELD OF THE INVENTION

The present invention is directed at a method of classifying an image feature in an electronic image processing system, in order to determine, from cardiac image data, a cardiac characteristic of a patient by using a trained machine learning data processing model, wherein the trained machine learning data processing model is trained to evaluate a probability that the image feature matches each class of a predetermined plurality of classes forming a set of classes such as to characterize the image feature, the cardiac image data comprising at least a region of interest including the image feature, wherein the method comprises: receiving, by the trained machine learning data processing model, the cardiac image data; obtaining, by the trained machine learning data processing model, data indicative of the region of interest; collecting, from the trained machine learning data processing model, probability data, the probability data comprising for each class of the set of classes a probability that the image feature matches the respective class. The invention is further directed at a method of training a machine learning data processing model for performing a method as described, and at an electronic image processing system.

### BACKGROUND

The current state of technology in medical imaging provides many different imaging techniques that together allow for detection and evaluation of many different characteristics or conditions, in an ever early stage. However, regardless of this, in many cases the proper evaluation still remains challenging. This may be for a large variety of reasons, for example: the imaged subject is in continuous motion, the obtainable resolution is not sufficient to reveal all necessary details, the size and shape of a subject renders it difficult to resolve or identify on image, the image available is two-dimensional and renders it difficult to do the evaluation, the differences in tissue structure provide a low contrast, etcetera.

An example wherein the evaluation of some health conditions is difficult to perform is in the field of cardiac imaging. The continuous motion of a living heart combined with the material parameters of the tissue, diminishes the image quality such that various characteristics or conditions cannot be detected with sufficient certainty from a cardiac image. In certain doubtful cases, a same image being evaluated by multiple physicians may not lead to a decisive conclusion due to disagreement between the physicians being fed by uncertainty on what an image really shows. This of course is not desired. Although image recognition based on artificial intelligence has improved this, it has not yet completely resolved the issue.

### SUMMARY OF THE INVENTION

It is an object of the present invention to ameliorate the evaluation of cardiac images, and provide a method and system for classifying an image feature thereof, in particular to facilitate decision making by a physician.

To this end, there is provided herewith in accordance with a first aspect of the invention, a method as described above, wherein the method further comprises, the steps of: evaluating, by a controller, the probability data for the classes of the set of classes, and identifying one or more candidate classes having a significant probability of matching with the image feature; determining, based on the step of evaluating, a certainty level indicative of a certainty that the image feature matches each of the one or more candidate classes of the set of classes; and dependent on the step of determining, performing at least one step of: when the number of candidate classes equals one, and the certainty level exceeds a threshold level: associating the one candidate class with the image feature; or when the number of candidate classes equals one, and the certainty level is smaller than a threshold level: requesting user input indicative of a confirmation or disaffirmation that the one candidate class matches with the image feature; or when the number of candidate classes exceeds one: requesting, for each of the one or more candidate classes, user input indicative of a confirmation or disaffirmation that the respective candidate class matches with the image feature.

The method of the present invention classifies image features, in cardiac image data, to match (or not match) a cardiac characteristic of a patient, and uses therefore a trained machine learning data processing model. In particular, the machine learning data processing model evaluates the probability of each class of a predetermined plurality of classes forming a set of classes in order to characterize the image feature. Thus, a closed set of predetermined classes is to be evaluated as to whether or not it is likely that they occur. The closed set will be mutually exclusive for the condition, i.e. if one of the classes applies, none of the other classes apply. In accordance with the present invention, the trained machine learning data processing model provides probability data, which for each class of the set of classes indicates a probability that the image feature matches the respective class. The probability data may for example provide a probability distribution which describes this probability, or alternatively the outcome thereof for the particular case. This class probability data may then be evaluated by the controller for each of the classes of the set of classes. One or more candidate classes may therefrom be identified, that have a significant probability of matching with the image feature. For example, a significant probability may be any of: an above average or mean probability as compared to probabilities of other (candidate) classes, a maximum probability or the top number of highest of probabilities (e.g. top three highest) of the various probabilities associated with each of a plurality of classes, a probability being significantly higher than the nearest probability below, or the 5^{th}, 10^{th}, 15^{th} or n^{th} percentile (0<n<50) of largest probabilities. The probability is indicative of a likelihood that the image feature matches a respective class. Based on a comparison between the probabilities obtained in the evaluation step, such as by performing a comparison of the probability data for the candidate classes, a certainty level is determined or estimated that is indicative of a certainty that the image feature matches each of the one or more candidate classes of the set of classes. It will be appreciated that for each of the identified candidate classes a certainty level is determined, wherein said certainty level is indicative of the certainty that the image feature matches the respective candidate class. The certainty level can be indicative of a likelihood that the image feature matches the candidate class relative to other candidate classes. The final outcome of the method will be dependent on the certainty level, and in accordance with the method is determined as follows. In this connection, the term ‘certainty level’ must be interpreted sufficiently broad to include a numeric or real value (e.g. a percentage or fraction), or a certainty class (e.g. ‘low’, ‘medium’, ‘high’; or 0%-10%, 10%-20%, 20%-30%, . . . , 90%-100%; or ‘very unlikely’, ‘unlikely’, ‘average’, ‘likely’, ‘very likely’).

Dependent on the step of determining the certainty level, in accordance with the method, when in the step of identifying candidate classes only a single candidate class remains (i.e. the number of candidate classes equals one) and the certainty level exceeds a threshold level, the one candidate class is associated with the image feature. The threshold level in that case should not leave any room for a different outcome. The threshold level may be dependent on various factors, and may be predetermined as a setting in the system. For example, the threshold level may be set to 70%, 75%, 80%, 85%, 90% or 95% (e.g. anything between 65% and 100%) dependent on preference. Furthermore, the threshold level does not have to be constant, but can be a non-linear function of the descriptive statistics of the probability distribution, e.g. determined by the controller or by another entity.

When, however, in accordance with the method the number of candidate classes equals one and the certainty level is smaller than a threshold level, the method may provide this as output to the user of the system and request input from the user. This input may be indicative of a confirmation or disaffirmation that the user (typically a physician) considers the one candidate class to match with the image feature. In certain embodiments, in accordance with the method the system may provide the probabilities or certainty levels to the user and/or may indicate which class is considered to provide the most likely outcome.

When, in accordance with the method, more than one candidate class may be possible (i.e. the number of candidate classes exceeds one), the method may continue with indicating this to the user and request user input. In this case, user input indicative of a confirmation or disaffirmation that the respective candidate class matches with the image feature is requested to the user.

The present invention improves the decision making process. Due to the fact that all classes of the closed set of mutually exclusive classes are evaluated against their probabilities of occurring based on the image data, a proper outcome may in more situations be decisively provided by the system. This diminishes the chance on an incorrect diagnosis. At the same time, the system does not steer the user in a wrong direction, because in those cases that are not completely certain this is flagged to the user and user input is requested. The system thus only improves the current situation, while preventing an incorrect conclusion or diagnose based on an (uncertain) appearance of a characteristic or condition. This is because the system is better able to judge whether it is indeed challenging or impossible to make a good judgement.

In some embodiments, the class probability data may be expanded with counter-class probability data, i.e. probability data that is indicative of a probability that the image feature does not match the respective class. Normally, the sum of class probability data and counter-class probability data will add up to 100% (this may optionally even be assumed), although this may not be per definition the case, as will be explained further below. The counter-class probability data may be used in the determination of the certainty level. If for example one candidate class appears to be most likely, then for each of the other classes the counter-class probability data must indicate to be most likely for that particular class (i.e. indicative of the class being not likely).

In many cases, the class probability data and the counter-class probability data may add up to 100%. However, in some cases, imperfections in the image (i.e. noise, disturbances, artefacts, etc.) may result in the sum of the class and counter-class probability data not to add up to 100%. The difference between this sum and 100%, in that case, may be indicative of image quality with respect to enabling evaluation of the respective class or may be indicative of the existence of one or more additional unknown classes. In images with insufficient image quality, wherein no clear and decisive probability on whether or not the image features matches the class can be provided, the method may signal this outcome to a user and also indicate that this is due to an insufficient quality of an image. This prevents the establishment of an incorrect conclusion or diagnose where a picture is indecisive.

In other or further embodiments, the method further comprises providing output to a user of the system, wherein the output comprises at least one element of a group comprising: data indicative of the one or more candidate classes; data of an association between one candidate class and the image feature; the certainty level; or the probability data. Typically, in accordance with the method, an association between a certain class and an image feature may be provided.

For example, considering a case of determining aortic valve morphology, the set of classes evaluated may include any one or more of: bicuspid aortic valve type 0 (BAV-0), bicuspid aortic valve type 1 (BAV-1), tricuspid aortic valve (TAV), quadricuspid aortic valve (QAV), pentacuspid aortic valve (PAV), unicuspid aortic valve (UAV) or n-cuspid aortic valve. Typically, the first three of these (BAV-0, BAV-1 and TAV) more frequently occur. The trained machine learning data processing model may be trained to consider the above mutually exclusive classes, and determine from a cardiac image at the input, whether any of the image features in the region of interest gives rise to an association with any of the above classes. Probability data indicative on the occurrence of each of these classes may be obtained from the parameters of the trained machine learning data processing model. Optionally, also probability data of counter-classes may be determined. The counter-classes are complementary to each class, e.g. for the class BAV-0 the counter-class is NOT_BAV-0 (the probability that the image feature is indicative of not BAV-0), for the class BAV-1 the counter-class is NOT_BAV-1 and for the class TAV the counter-class is NOT_TAV, etc. Once an image feature in accordance with the method can be associated with a class (or a counter-class), this association may be presented as output of the system to the user. Also, optionally the certainty level for this may be presented as output, and if desired also the probability data for each class (and/or counter-class) may be presented as output.

In some embodiments, for providing the probability data, the machine learning data processing model comprises at least one Gaussian layer in order to determine for each class of the set of classes said probability that the image feature matches the respective class. For example, the use of a Gaussian layer in an artificial neural network directly enables to obtain probability data for a certain outcome. This may be obtained for each class (and optionally each counter-class). Alternatively, similar results may be obtained by applying a Bayesian network, which provides a good alternative for obtaining probability data.

In some embodiments, the trained machine learning data processing model is a convolutional neural network including one or more intermediate layers, and wherein the step of collecting further comprises: obtaining from at least one of the intermediate layers, a weighting vector of M weights and intermediate data of M channels, wherein M comprises a natural number larger than zero; weighting the intermediate data using the weights resulting in weighted intermediate data; and sampling the weighted intermediate data resulting in region-indicating data, the region-indicating data being indicative of one or more regions of the cardiac image having an above average weighing in the weighted intermediate data.

In other or further embodiments, the trained machine learning data processing model comprises an input layer for receiving at least the cardiac image data, and an output layer for providing output data, the output data including at least one of: data indicative of the one or more candidate classes, an association between one candidate class and the image feature, the certainty level, or the probability data; wherein the method further comprises the steps of: modifying, by the controller, one or more parts of the cardiac image data; evaluating, by the trained machine learning data processing model, a change in the output data in response to the step of modifying, wherein the change relates to at least one of the candidate classes, the association, the certainty level, or the probability data; and identifying, based on the step of evaluation, one or more regions of significance in the cardiac image data; and providing region-indicating data, wherein the region-indicating data is indicative of the one or more regions of significance.

The above two groups of embodiments, which may be referred to as class activation mapping, provide significant advantages, because they enable to provide more insight in the image features and regions of an image that support a certain outcome. For example, certain image features may be more relevant to a certain outcome. By analysis of the weights in the above manner, it becomes apparent which image features are of greater weight in a certain outcome (e.g. why presence of certain image features indicates that a certain class does not appear to apply, or vice versa). These image features or regions in the image may then be highlighted in the output of the system, such as to present these to the user. In particular in those outcomes wherein the user is requested to provide further input to confirm or disaffirm the outcome, this may be helpful. However, also in the cases where the outcome is clear, the highlighting of these features may provide valuable information to the user. Therefore, in some of the above embodiments, the method further comprises providing output to a user of the system, wherein the output comprises the region-indicating data.

In yet further embodiments, the step of sampling comprises at least one of: up-sampling, such as bilinear up-sampling or interpolation; down-sampling, such as decimation. Up-sampling or down-sampling may be desired in order to match the resolution of the region-indicating data to that of the image. It may also be applied to locally improve the image resolution, for example in order to add auxiliary image features or markers, e.g. indicators, explanatory text, weighting values, etc.

In some embodiments, the step of obtaining the data indicative of the region of interest comprises at least one of: obtaining user input indicative of the region of interest; or performing a pre-processing filter operation for establishing the data indicative of the region of interest; or performing, using the or a further trained machine learning data processing model, a pre-processing image recognition step for establishing the data indicative of the region of interest. A region of interest may be obtained in various different ways. As may be appreciated, a user may provide a region of interest as input to the system. For example, the user may be interested in a specific region of an image wherein a lesion or defect may be suspected, or which is otherwise of interest for evaluation. However, in some embodiments, a pre-processing filter operation may automatically establish a region of interest. This could be useful in those cases wherein a region of interest is relatively well to establish using a computer implemented method or electronic means, for example: by applying an algorithm; by considering a specific part of a spectrum; by performing contour recognition based on contrast, or based on color or texture differences; or because a certain region of interest may always be found in a specific part of an image (this may be a fixed part of the image (e.g. always in the upper left corner, or in the middle) or in relation to other features (e.g. always at a certain location between two well recognizable lung areas). However, in some embodiments, the region of interest may be established performing, using the or a further trained machine learning data processing model, a pre-processing image recognition step for establishing the data indicative of the region of interest. Here, image recognition may be performed by the data processing model in order to perform the automatic step. Each of the latter two automatic processes provides the advantage of ease of use of the system, and further enables to perform the analysis for a series of images containing cardiac image data. For example, a collection of cardiac image data from a database may be examined in this manner in order to establish statistics on a certain physical condition, such as statistics on the occurrence of specific types of left atrial appendage (LAA) morphology: chicken-wing, windsock, cauliflower, and cactus.

In some embodiments, the cardiac image data comprises one or more of: computed tomography scan data, positron emission tomography data, magnetic resonance imaging data, ultrasound imaging data such as echocardiographic imaging data, radiographic imaging data, angiographic imaging data. In fact, the invention may be applied to any imaging modality, and the abovementioned modalities are mentioned mainly as examples.

In yet further embodiments, the region of interest is related to an atrium of the patient and the classes of the set of classes comprise aortic valve morphology labels. For example, in some embodiments, the aortic valve morphology labels comprise at least one or more of: a bicuspid aortic valve type 0 (BAV-0) label, a bicuspid aortic valve type 1 (BAV-1) label, a tricuspid aortic valve (TAV) label, quadricuspid aortic valve (QAV) label, pentacuspid aortic valve (PAV) label, unicuspid aortic valve (UAV) label, or n-cuspid aortic valve label.

In other or further embodiments, the method may be applied in order to evaluate various other characteristics and conditions. For example, in a method in accordance with such applications, one or more of the following may apply. In some embodiments, the region of interest is related to a left atrial appendage and the classes of the set of classes comprise left atrial appendage morphology labels, such as a cowley flower label, a chicken wing label, a windsock label or a cactus label. In a different application, the region of interest is related to a mitral valve of the patient and the classes of the set of classes comprise calcification grade labels, such as wherein the calcification grade labels comprise at least a mild label, a moderate label and a severe label. In these embodiments, other or further labels may be applied, such as percentage classes or certain ranges that indicate seriousness. In yet other embodiments, the region of interest is related to an aortic root of the patient and the classes of the set of classes comprise calcification grade labels, such as wherein the calcification grade labels comprise at least a mild label, a moderate label and a severe label. Also in these embodiments, other or further labels may be applied, such as percentage classes or certain ranges that indicate seriousness. In other or further embodiments, the cardiac characteristic relates to a cardiac phase and the classes of the set of classes comprise percentage intervals or percentage labels. In other or further embodiments, the region of interest is related to a left atrial appendage (LAA) and the cardiac characteristic relates to a left atrial appendage filling, wherein the classes of the set of classes comprise percentage intervals or percentage labels.

In accordance with a second aspect of the invention, there is provided herewith a method of training a machine learning data processing model for performing a method according to the first aspect, for classifying an image feature in order to determine, from cardiac image data, a cardiac characteristic of a patient by evaluating a probability that the image feature matches each class of a predetermined plurality of classes forming a set of classes such as to characterize the image feature, the method comprising: a) receiving, by the machine learning data processing model, a training data set comprising training data, the training data including cardiac image data of at least a part of a cardiac image, and optionally data indicative of a region of interest which includes the image feature; b) obtaining, by the machine learning data processing model, a plurality of classes forming a set of classes, wherein the classes together describe status classes of the cardiac characteristic; c) receiving, by the machine learning data processing model, a ground truth data set comprising ground truth data indicative of a ground truth class that matches with the image feature; d) training the machine learning data processing model based on the training data received in step a), the classes of the set of classes obtained in step b), and the ground truth data received in step c), for enabling the machine learning data processing model, after completion of the training period, to perform the step of providing probability data, the probability data comprising for each class of the set of classes a probability that the image feature matches the respective class.

The above method in accordance with the second aspect, enables to train a machine learning data processing model in order to use such a trained model in a method in accordance with the first aspect. It will therefore be appreciated that the method in accordance with the second aspect enables to train the machine learning data processing model, and the trained machine learning data processing model is configured for performing a method according to the first aspect. The machine learning data processing model that may be trained using this method may be a neural network that includes a Gaussian layer, such as an artificial neural network or a convolutional neural network including a Gaussian layer, or a Bayesian network. These data processing models enable to provide probability date together with potential outcomes, such as applied in the method of the present invention.

In accordance with a third aspect of the invention, there is provided herewith an electronic image processing system for use in a method according to the first aspect, the system comprising a trained machine learning data processing model according to the second aspect, for classifying an image feature in order to determine, from cardiac image data, a cardiac characteristic of a patient by evaluating a probability that the image feature matches each class of a predetermined plurality of classes forming a set of classes such as to characterize the image feature, wherein the system further comprises one or more processors and a memory storing the trained machine learning data processing model and one or more instructions, which when loaded into the memory enable the one or more processors to perform the steps of: receiving, by the trained machine learning data processing model, the cardiac image data; obtaining, by the trained machine learning data processing model, data indicative of the region of interest; collecting, from the trained machine learning data processing model, probability data, the probability data comprising for each class of the set of classes a probability that the image feature matches the respective class; wherein the method further comprises, the steps of: evaluating, by a controller, the probability data for the classes of the set of classes, and identifying one or more candidate classes having an above average probability of matching with the image feature; determining, based on the step of evaluating, a certainty level indicative of a certainty that the image feature matches each of the one or more candidate classes of the set of classes; and dependent on the step of determining, performing at least one step of: when the number of candidate classes equals one, and the certainty level exceeds a threshold level: associating the one candidate class with the image feature; or when the number of candidate classes equals one, and the certainty level is smaller than a threshold level: requesting user input indicative of a confirmation or disaffirmation that the one candidate class matches with the image feature; or when the number of candidate classes exceeds one: requesting, for each of the one or more candidate classes, user input indicative of a confirmation or disaffirmation that the respective candidate class matches with the image feature.

### BRIEF DESCRIPTION OF THE DRAWINGS

The invention will further be elucidated by description of some specific embodiments thereof, making reference to the attached drawings. The detailed description provides examples of possible implementations of the invention, but is not to be regarded as describing the only embodiments falling under the scope. The scope of the invention is defined in the claims, and the description is to be regarded as illustrative without being restrictive on the invention. In the drawings:

FIG. 1 schematically illustrates the artificial neural network for performing image recognition, in accordance with the prior art;

FIG. 2 schematically illustrates a neural network including a Gaussian process layer for adding probability data to an image recognition process;

FIG. 3 schematically illustrates the application of trained machine learning data processing model in an embodiment of the present invention;

FIG. 4 schematically illustrates an embodiment of the method of the present invention;

FIG. 5 schematically illustrates an embodiment of the method and system of the present invention;

FIG. 6 schematically illustrates a method of the present invention in accordance with an embodiment thereof; and

FIG. 7 schematically illustrates an electronic image processing system in accordance with an embodiment of the present invention.

### DETAILED DESCRIPTION OF THE DRAWINGS

Terminology used for describing particular embodiments is not intended to be limiting of the invention. As used herein, the singular forms “a”, “an” and “the” are intended to include the plural forms as well, unless the context clearly indicates otherwise. The term “and/or” includes any and all combinations of one or more of the associated listed items. It will be understood that the terms “comprises” and/or “comprising” specify the presence of stated features but do not preclude the presence or addition of one or more other features. It will be further understood that when a particular step of a method is referred to as subsequent to another step, it can directly follow said other step or one or more intermediate steps may be carried out before carrying out the particular step, unless specified otherwise. Likewise it will be understood that when a connection between structures or components is described, this connection may be established directly or through intermediate structures or components unless specified otherwise.

The invention is described more fully hereinafter with reference to the accompanying drawings, in which embodiments of the invention are shown. In the drawings, the absolute and relative sizes of systems, components, layers, and regions may be exaggerated for clarity. Embodiments may be described with reference to schematic and/or cross-section illustrations of possibly idealized embodiments and intermediate structures of the invention. In the description and drawings, like numbers refer to like elements throughout. Relative terms as well as derivatives thereof should be construed to refer to the orientation as then described or as shown in the drawing under discussion. These relative terms are for convenience of description and do not require that the system be constructed or operated in a particular orientation unless stated otherwise.

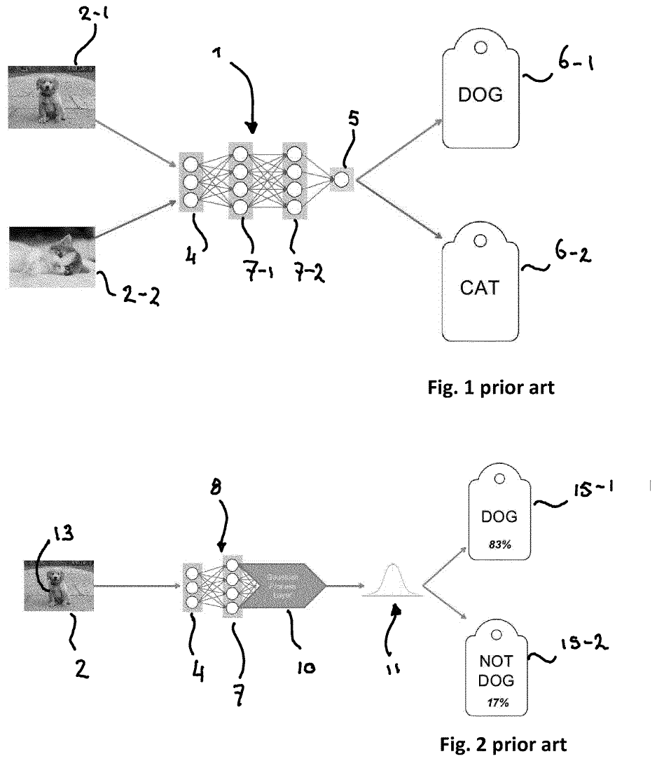

In FIG. 1, a conventional artificial neural network 1 for performing image recognition is schematically illustrated. The neural network 1 comprises an input layer 4 and an output layer 5. At the input layer 4, the neural network 1 may receive various images 2-1 and 2-2. The neural network 1 in FIG. 1 has been trained to perform image recognition, and is able to recognize the subjects shown on each of the images 2-1 and 2-2 as animals and determine the type of animal. In particular, the neural network 1 may be trained to either identify the animal on the picture 2-1 or 2-2 as a dog or a cat. To enable the neural network 1 to perform this task, the neural network 1 may have undercome a training program. The training program for example could have been a supervised training program. In such a training program. A plurality of various images 2 of dogs and cats may have been offered to the neural network 1 together with ground truth data. The ground truth data in this case provides the correct classification, i.e. the classification as provided or confirmed by an operator or user. During training, the images 2 may be provided to the neural network 1 via the input layer 4. In a first phase, the ground truth data may be actively used to adapt and optimize the parameters in the various layers (e.g. intermediate layers 7) of the neural network 1 such as to provide the correct animal type classification: ‘dog’ or ‘cat’. In a second phase of the training, the neural network 1 receives the images 2 (such as 2-1 and 2-2) via the input layer 4 and provides a classification at the output layer 5. These classifications of the images 2 at the output 5 are then verified against the ground truth data. Based on a comparison of the results 6-1 and 6-2 coming from the output layer 5 and the ground truth data, the parameters in each of the intermediate layers 7-1 and 7-2 and their interconnections may be altered to train the network. Dependent on whether the classes provided as results 6-1 and 6-2 are correct, the neural network 1 may be optimized. After training, the conventional neural network 1 is then able to classify an animal on an arbitrary image 2 as being either a ‘cat’ or a ‘dog’.

Although neural network 1 at the output layer 5 may provide impressive recognition results after proper training, the neural network 1 of FIG. 1 does not provide any insight into how certain this result is, or in other words, how likely it is that the neural network 1 provides an incorrect class as result 6 for an image 2. This in particular will become an issue in those areas wherein the differences between elements of two or more classes are only marginal, and where at the same time the risks involved in making an incorrect assessment may be significant. So, in the example of distinguishing between cats and dogs in arbitrary images the risks may be absent. However, the risks involved in distinguishing between classes in a medical setting may be significant, in particular when potentially life threatening physical conditions are involved.

FIG. 2 illustrates another neural network 8 comprising an input layer 4, various intermediate layers 7 and a Gaussian process layer 10. Gaussian processes are a generalization of the Gaussian probability distribution. Gaussian process regression calculates the probability distribution over all admissible outcomes. The Gaussian process layer 10 thus enables the neural network 8 to provide after training, with each outcome, probability data. The probability data provided is indicative of a probability that the class provided with the animal in the image 2, a dog, is indeed correct. To do this, one of the intermediate layers 7 of the neural network 8 interconnects with the Gaussian process layer 10, and the intermediate results from the intermediate layer 7 are processed to provide the probability in accordance with a Gaussian profile 11. At the output of neural network 8, the image feature 13 is not only classified as a dog 15-1, but also probability data is provided which indicates the probability that the image feature 13 is indeed a dog. In the example, the available classes are either the class ‘dog’ 15-1, or the counter class ‘not dog’ 15-2.

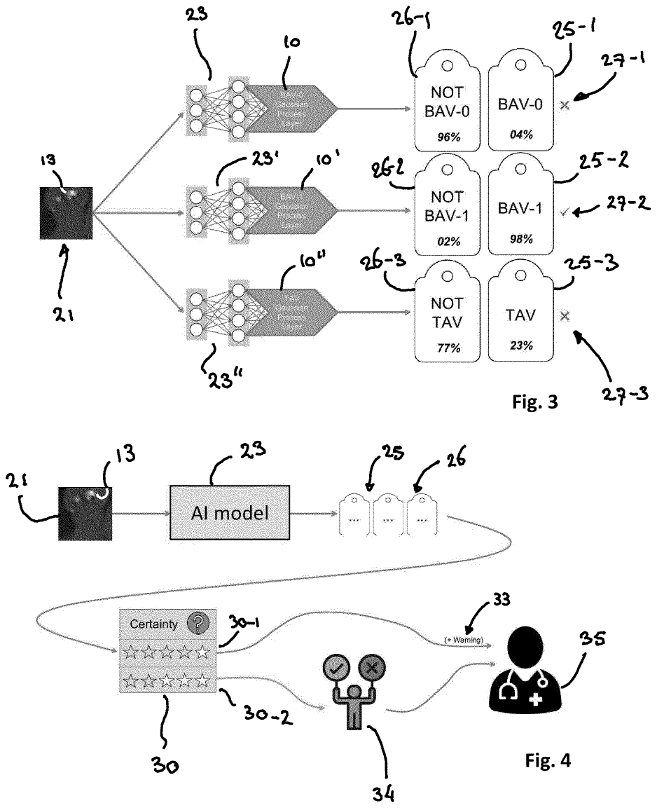

The present invention applies one or more Gaussian process layers 10 in a trained machine learning data processing model 23 (e.g. FIG. 3) that enables the classifying of image features from a cardiac image in order to determine therefrom a cardiac characteristic of a patient. For certain cardiac characteristics, the relevant image features 13 of images 21 obtained from an imaging system 80 (see FIG. 7) do not always provide 100% certainty as to how the imaged cardiac characteristic is to be classified. In these cases, making an incorrect classification may provide a direct serious health risk to the patient. The method and system of the present invention support a proper conclusion or diagnose by taking part of the uncertainty away, or otherwise making the user 35 (see FIG. 4) aware of this uncertainty and the level of uncertainty. In FIG. 3, the method of the present invention, which involves the use of a trained machine learning data processing model 23 is schematically illustrated. In the method illustrated in FIG. 3, the machine learning data processing model 23 is illustrated three times as 23, 23′ and 23″. In principal it is possible to use three different neural networks with Gaussian process layers 10, 10′ and 10″ that are each trained to evaluate a specific class (e.g. the bicuspid aortic valve type 0 (BAV-0)′) and its counter-class (in that case ‘NOT BAV-0’), in order to for the combination of data processing models 23, 23′ and 23″ to allow an evaluation as to whether or not the image feature illustrated in image 21 belongs to a specific class 25-1 (or 25-2 or 25-3), or its counter class 26-1 (or 26-2 or 26-3). However, this may also be achieved by training a single neural network 23 with one or more Gaussian layers 10 to perform the classification over all classes 25-1, 25-2 and 25-3 and/or their counter classes 26-1, 26-2 and 26-3. The classes in FIG. 3 are mutually exclusive, meaning that if the condition illustrated in image 21 is indeed of bicuspid aortic valve type 0 (BAV-0), it cannot be bicuspid aortic valve type 1 (BAV-1) nor tricuspid aortic valve (TAV). This is not a necessity for the method of the present invention, as one may appreciate it is also possible to add another class which is indicative of e.g. a severeness of a certain condition or a comorbidity. However, in order to not unnecessarily complicate the example, we will assume here that the classes 25-1, 25-2 and 25-3 are mutually exclusive. For each class 25-1 through 25-3, there is a counter class 26-1 through 26-3 indicative of the image feature not belonging to the respective class.

The system of FIG. 3 and its output provides three classes 25-1 through 25-3 and three counter classes 26-1 through 26-3, including their probability data of whether or not the class or counter class applies to the respective image feature. For example in image 21 of FIG. 3, there is a 96% probability that the image feature does not belong to the class BAV-0 indicative of an aortic valve morphology of bicuspid aortic valve type 0. The system provides a 98% probability that the image feature in image 21 belongs to the class bicuspid aortic valve type 1 (BAV-1). Furthermore, the system indicates that there is a 23% probability that the image feature in image 21 belongs to the class tricuspid aortic valve (TAV). Based on the probability data in each of the classes 25-1 through 25-3 and each of the counter classes 26-1 through 26-3, it is relatively certain that the image feature is visible in image 21 belongs to this particular class BAV-1. This will therefore be the result 27-2 presented at the output of FIG. 3. Other possibilities 27-1 and 27-3 are discarded. To perform this step, the system in FIG. 3 does determine a certainty level which is indicative of a certainty that the image feature in image 21 matches each of the classes 25 or counter classes 26, and on basis of that, because there is only as single candidate class (BAV-1) having a certainty level of 98%, whereas the nearest alternative has a probability of 23% (TAV). The difference between these two being 75% exceeds a preset threshold level (which may be set to any desired value between 1% and 100%, e.g. 20%, 30%, 40%, 50%, 60%, 65%, 70%, 75%, 80%, 85%, 90% or 95%), and therefore the system is able to directly associate the image feature in image 21 with the candidate class BAV-1. Alternatively, the system may also be programed to select the class having the highest probability if that probability at least is above a predetermined threshold level (for example a value in a range between 60% and 100%, or 80% and 100%). This of course may not always be the case.

Suppose the probability data in FIG. 3 would have been different, and suppose that the probability for class BAV-1 would have been 60% whereas the probability for class BAV-0 would have been 40%, it is still more likely that class BAV-1 will apply to the image feature of image 21, but the certainty level is much lower. The difference between probability is only 20% and the highest probability is at 60%. Therefore, the certainty level which is indicative of how certain it is that the suggested class at the output 5 is the correct class, may be below a predetermined threshold level. The threshold level may for example be any of the above, e.g. associated with a difference in the two (or more) highest probabilities or the minimal probability value of the class having the highest probability. Suppose such a probability difference threshold level would be set at a minimum of 30%, the certainty level in this example is not sufficient. The certainty level and the threshold in the invention serve as a safety measure in the system. If, as in this case, the certainty level is insufficient, a user 35 may be consulted to confirm or disaffirm the result, i.e. whether or not the class BAV-1 is indeed correct. This is illustrated in FIG. 4.

In FIG. 4, image 21 is offered at the input of the trained machine learning data processing model 23 (our convolutional neural network with Gaussian process layer 10). The data processing model 23 provides the classes 25 and counter classes 26 including probability data, as is illustrated in FIG. 3. Thereafter, the certainty level 30 of the result coming from data processing model 23 is determined. In the example of FIG. 4, the certainty level is represented as a number of stars between zero and five stars. The threshold level in such embodiments may likewise be set as a number of stars (e.g. three stars). If there is a sufficiently high certainty level as in situation 30-1, the method continues by providing the result of the classification directly to the user 35. This presentation of the result may include a warning 33 to the user 35, that the recognition is performed using an artificial intelligence model, and not by a physician. If the certainty level 30-2 is insufficient (here below three stars), user input 34 is requested in order to confirm or disaffirm that the candidate class suggested by the system 23 matches with the image feature 21. In that manner, the user 35 which may be a physician is informed of the fact that the system 23 considers the image feature to belong to a certain class, but the physician is also informed of the fact that the system 23 is not sufficiently certain about this. This therefore flags to physician 35 that additional care should be taken in evaluating this image or taking over the class 25 suggested by the system.

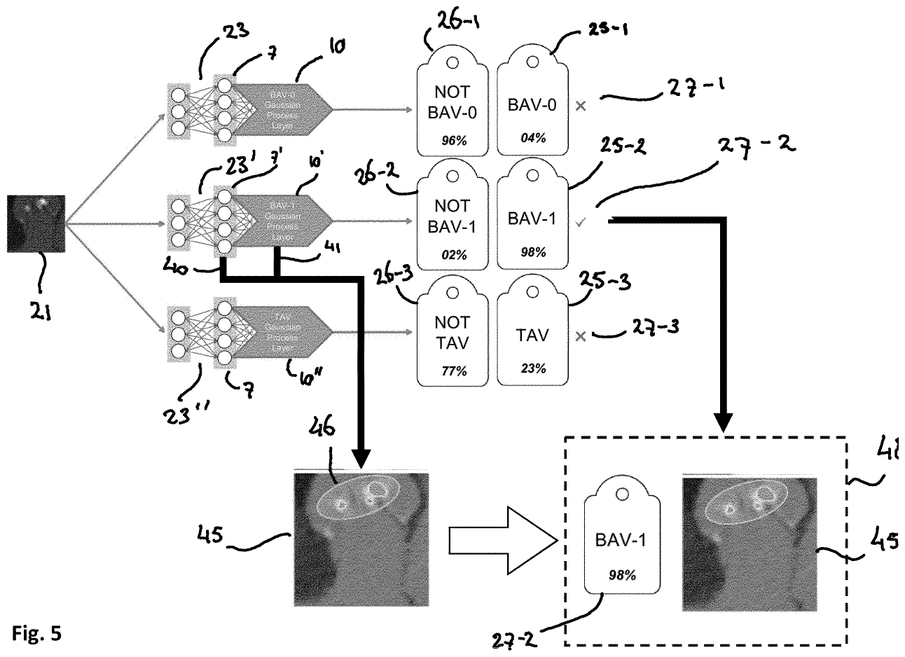

FIG. 5 illustrates another embodiment of the method and system of the present invention, which provides the user 35 with even more information on the selected class. In FIG. 5, the system of FIG. 3 is schematically illustrated including again the machine learning data processing model 23, 23′ and 23″. In FIG. 5 a class activation mapping process is performed by analyzing the parameter values from intermediate layers 7′ in step 40, as well as analyzing the Gaussian layer 10′ in step 41. In particular, the class activation mapping is performed by obtaining from the intermediate layer 7′ a weighting factor of a respective channel or interconnection present in the intermediate layer 7′ and the intermediate data of N-channels in the intermediate layer 7′. The intermediate data from the channels is weighted using the weights of the intermediate layer 7′, and optionally the data from the Gaussian process layer 10′ may be used in order to identify the weighted intermediate data having a significant effect (e.g. the most effect) on the outcome of the classification process. This data may then be mapped onto the image 21, which yields the mapping 45 to include region indicating data, wherein those parts of the image 21 that are most significant in determining the resulting class have been highlighted. The mapping 45 is provided together with a result 27-2 and probability data as the outcome 48 of the system.

FIG. 5 shows one category of embodiments that enables implementation of class activation mapping in the method of the invention. However, the invention is not limited to this particular implementation. Alternatively, class activation mapping may also be performed by examining the outcome of the model on characteristics of the input. For example by modifying input characteristics (e.g. relating to parts of a cardiac image) and evaluating how strongly the output responds to a change at the input. Such a method may for example be perturbation or occlusion-based. These methods evaluate how a predicted output class, segmentation or any of the intermediate outputs (feature maps/activation maps) change in response to occlusion or perturbation of a certain region. The magnitude of change relates to the importance of a given region. The region can be color-coded accordingly to yield visualizations. In this case no rescaling/sampling has to take place, the regions are defined in the original input image/volume. Examples are SHAP or LIME. Alternatively, same may be achieved based on or using saliency maps. These methods take a look at the gradient of the classification loss with respect to the input pixels/voxels. Highly salient pixels/voxels need to change the least to affect the class prediction the most. No rescaling/sampling has to take place here, a value is obtained for each pixel/voxel individually. There are extensions that multiply the gradients by the input value (‘gradient*input’) or that combine saliency maps with perturbation/occlusion-based methods. For instance, ‘integrated gradients’ compute the gradients for each input pixel/voxel in a set of increasingly less perturbed versions of the same image, as show in in the example below. The average of the input pixel/voxel gradients is computed to get the final result.

Back to FIG. 5 (although the below also applies to the alternatives, mutatis mutandis), the class activation mapping provides the user 35 with additional information as to how the system 23 came to the conclusion that the respective image 21 shows a BAV-1 type of aortic valve morphology. This enables the user 35 to evaluate this choice, and particular where the certainty level is low, allows the user 35 to reconsider this choice of class.

The above method illustrated in FIG. 5 is based on analysis of the values of the convolutional neural network. A convolutional neural network consists of a series of layers. An input image passes through the first layer, which results in feature maps. Next, some kind of pooling operation can take place which can reduce the dimensionality of the feature maps. The feature map gets passed on to other layers resulting in new feature maps at increasingly coarser resolutions. In a traditional CAM implementation, the penultimate feature maps are used, along with the weights of the final layer. The weights that are connected to the predicted class are multiplied with the different channels of the penultimate feature maps. These are the weights that resulted in the highest probability for this particular image to belong to class c. The result of the weighting is a class activation map which typically is of coarser resolution than the original image. To get a result in the original resolution, some resizing/upsampling has to take place. Note that in this implementation, an approach is used with GAP (global average pooling) which reduces each of the N feature maps to a single average value. These single values are multiplied with weights, resulting in N weights. Grad-CAM: Grad-CAM differs from CAM in the sense that is does not use raw layer weights, but rather uses the gradients. Grad-CAM is more flexible and computes the gradients of the classification loss for predicted class c with respect to each element of each feature map. An averaging step takes place over each feature map to give a single weighting value for each feature map. The average of the averaged feature is computed to give a visual output. Extensions exist to this approach that compute gradients on perturbed feature maps, or that combine CAM-like methods with saliency-based methods.

It is stressed that, although the above examples have been directed at the application of determining a type of aortic valve morphology, this is certainly not the only field of application wherein the above may be applied. The invention may likewise be applied in relation to different physical cardiac characteristics. For example, in the classification of left atrial appendage morphology, or calcification grade of an aortic root or a mitral valve. For example, the left atrial appendage morphology classes to be evaluated may include any one or more or all of: a Cowley flower label, a chicken wing label, a windsock label or a cactus label. Instead or in addition, the classes may relate to parameter ranges, percentages, quantitative or qualitative descriptors, or the like. For example to express a calcification grade, it is possible to classify this as mild, moderate or severe, or otherwise e.g. in terms of vessel blocking percentage (e.g. percentage classes) or size range classes.

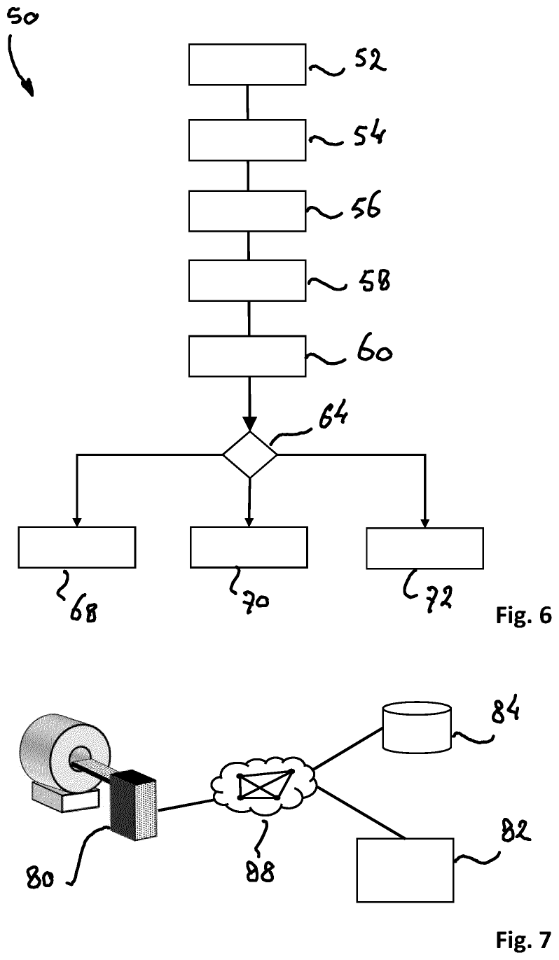

FIG. 6 schematically illustrates a method in accordance with the present invention. In FIG. 6, a trained machine learning data processing model 23 receives cardiac image data 21 in step 52. For example this may be a cardiac image 21 illustrated in FIG. 3. In step 54 of method 50, the trained machine learning data processing model 23 may further receive data which is indicative of a region of interest. For example, user 35 may indicate that she/he is interested in a region that illustrates the aortic valve in order to make an assessment on the aortic valve morphology. The user 35 may simply indicate the respective region by indicating or marking the contours thereof. Alternatively, if possible, an automatic algorithm or preprocessing filter operation may be performed in order to identify the region of interest and provide this to the system 23. As a further alternative, it is also possible to apply a further trained machine learning data processing model which is able to recognize a specific region of interest in a cardiac image and automatically provide the region of interest to the trained machine learning data processing model 23. For example a further trained machine learning data processing model may be able to recognize the location of the aortic valves, or any of the cardiac atriums, or the aortic root valve or the like.

In step 56, from the trained machine learning data processing model 23, probability data is collected from the Gaussian process layer 10. This probability data provides, for each class 25 of the set of classes to be considered, a probability that the image feature matches respective class. Optionally, it is further possible to add counter classes 26 to each class 25, indicative of the probability that an image feature does not match a specific class.

In step 58, the outcome from trained machine learning data processing model 23 is evaluated by the controller. The controller evaluates probability data for each class 25 of the set of classes, and from this identifies one or more classes having an above averaged probability of matching with the specific image feature. Then, in step 60 a certainty level is determined on the basis of the probability data for each of the identified classes. The certainty level is indicative of the certainty that the image feature matches a certain preferred class 25 of the set of classes. For example, the certainty level may be high or low dependent on whether or not the probability data provides a large probability for certain class and thereby a large difference between probabilities of the preferred class with respect to the other classes. In step 64, the outcome of the determining step is evaluated. In case where there is only one candidate class left, and the certainty level is high, such as in FIG. 3, the method continuous in step 68 by directly associating this one candidate class with the image feature from the image 21. Alternatively, in step 70, if there is one candidate class and the certainty level is below a threshold level (for example a probability of 60% in favor of the class, while the probabilities of the other classes are around 30%), the system requests user input in order to indicate whether the preferred class is confirmed or disaffirmed by the user 35. In any case where there is more than one candidate class left, in step 72 the user 35 is requested, for each of these candidate classes, to indicate a confirmation of disaffirmation that the respective class 25 matches with the image feature.

FIG. 7 schematically illustrates a system in accordance with the present invention. In FIG. 7, an imaging system 80 is schematically illustrated which is connected to an electronic image processing system 82 via a data network 88. The data network 88 may be a local area network of a hospital, for example. Furthermore, the images from the imaging system 80 may have been stored in the database 84, from which the electronic image processing system 82 may also retrieve images 21 in order to perform the processing thereof using the method of the present invention.

The present invention has been described in terms of some specific embodiments thereof. It will be appreciated that the embodiments shown in the drawings and described herein are intended for illustrated purposes only and are not by any manner or means intended to be restrictive on the invention. It is believed that the operation and construction of the present invention will be apparent from the foregoing description and drawings appended thereto. It will be clear to the skilled person that the invention is not limited to any embodiment herein described and that modifications are possible which should be considered within the scope of the appended claims. Also kinematic inversions are considered inherently disclosed and to be within the scope of the invention. Moreover, any of the components and elements of the various embodiments disclosed may be combined or may be incorporated in other embodiments where considered necessary, desired or preferred, without departing from the scope of the invention as defined in the claims.

In the claims, any reference signs shall not be construed as limiting the claim. The term ‘comprising’ and ‘including’ when used in this description or the appended claims should not be construed in an exclusive or exhaustive sense but rather in an inclusive sense. Thus the expression ‘comprising’ as used herein does not exclude the presence of other elements or steps in addition to those listed in any claim. Expressions such as “consisting of”, when used in this description or the appended claims, should be construed not as an exhaustive enumeration but rather in an inclusive sense of “at least consisting of”. Furthermore, the words ‘a’ and ‘an’ shall not be construed as limited to ‘only one’, but instead are used to mean ‘at least one’, and do not exclude a plurality. Features that are not specifically or explicitly described or claimed may be additionally included in the structure of the invention within its scope. Any of the claimed or disclosed devices or portions thereof may be combined together or separated into further portions unless specifically stated otherwise, without departing from the claimed invention. Expressions such as: “means for . . . ” should be read as: “component configured for . . . ” or “member constructed to . . . ” and should be construed to include equivalents for the structures disclosed. The use of expressions like: “critical”, “preferred”, “especially preferred” etc. is not intended to limit the invention. Additions, deletions, and modifications within the purview of the skilled person may generally be made without departing from the spirit and scope of the invention, as is determined by the claims. The invention may be practiced otherwise then as specifically described herein, and is only limited by the appended claims.

The claims are amended as follows:

1. In an electronic image processing system, a method of classifying an image feature in order to determine, from cardiac image data, a cardiac characteristic of a patient by using a trained machine learning data processing model, wherein the trained machine learning data processing model is trained to evaluate a probability that the image feature matches each class of a predetermined plurality of classes forming a set of classes such as to characterize the image feature, the cardiac image data comprising at least a region of interest including the image feature, wherein the method comprises:
receiving, by the trained machine learning data processing model, the cardiac image data;
obtaining, by the trained machine learning data processing model, data indicative of the region of interest;
collecting, from the trained machine learning data processing model, probability data, the probability data comprising for each class of the set of classes a probability that the image feature matches the respective class;
wherein the method further comprises, the steps of:
evaluating, by a controller, the probability data for the classes of the set of classes, and identifying one or more candidate classes having a significant probability of matching with the image feature;
determining, by performing a comparison of the probability data for the candidate classes, for each of the identified candidate classes a certainty level indicative of a certainty that the image feature matches the respective candidate class; and
dependent on the step of determining, performing at least one step of:
when the number of candidate classes equals one, and the certainty level exceeds a threshold level: associating the one candidate class with the image feature; or
when the number of candidate classes equals one, and the certainty level is smaller than a threshold level: requesting user input indicative of a confirmation or disaffirmation that the one candidate class matches with the image feature; or
when the number of candidate classes exceeds one: requesting, for each of the one or more candidate classes, user input indicative of a confirmation or disaffirmation that the respective candidate class matches with the image feature.

2. The method according to claim 1, further comprising providing output to a user of the system, wherein the output comprises at least one element of a group comprising: data indicative of the one or more candidate classes; data of an association between one candidate class and the image feature; the certainty level; or the probability data.

3. The method according to claim 1, wherein for providing the probability data, the trained machine learning data processing model comprises a Gaussian layer in order to determine for each class of the set of classes said probability that the image feature matches the respective class.

4. The method according to any one or more of the preceding claim 1, wherein the trained machine learning data processing model is a convolutional neural network including one or more intermediate layers, and wherein the step of collecting further comprises:
obtaining from at least one of the intermediate layers, a weighting vector of M weights and intermediate data of M channels, wherein M comprises a natural number larger than zero;
weighting the intermediate data using the weights resulting in weighted intermediate data; and
sampling the weighted intermediate data resulting in region-indicating data, the region-indicating data being indicative of one or more regions of significance of the cardiac image having an above average weighing in the weighted intermediate data.

5. The method according to claim 1, wherein the trained machine learning data processing model comprises an input layer for receiving at least the cardiac image data, and an output layer for providing output data, the output data including at least one of: data indicative of the one or more candidate classes, an association between one candidate class and the image feature, the certainty level, or the probability data;
wherein the method further comprises the steps of:
modifying, by the controller, one or more parts of the cardiac image data; evaluating, by the trained machine learning data processing model, a change in the output data in response to the step of modifying, wherein the change relates to at least one of the candidate classes, the association, the certainty level, or the probability data; and
identifying, based on the step of evaluation, one or more regions of significance in the cardiac image data; and
providing region-indicating data, wherein the region-indicating data is indicative of the one or more regions of significance.

6. The method according to claim 4, further comprising providing output to a user of the system, wherein the output comprises the region-indicating data.

7. The method according to claim 4, wherein the step of sampling comprises at least one of: up-sampling, such as bilinear up-sampling or interpolation; down-sampling, such as decimation.

8. The method according to claim 1, wherein the step of obtaining the data indicative of the region of interest comprises at least one of:
obtaining user input indicative of the region of interest; or
performing a pre-processing filter operation for establishing the data indicative of the region of interest; or
performing, using the or a further trained machine learning data processing model, a pre-processing image recognition step for establishing the data indicative of the region of interest.

9. The method according to claim 1, wherein the cardiac image data comprises one or more of: computed tomography scan data, positron emission tomography data, magnetic resonance imaging data, ultrasound imaging data such as echocardiographic imaging data, radiographic imaging data, angiographic imaging data.

10. The method according to claim 1, wherein the region of interest is related to an atrium of the patient and the classes of the set of classes comprise aortic valve morphology labels.

11. The method according to claim 10, wherein the aortic valve morphology labels comprise at least one or more of: a bicuspid aortic valve type 0 label, a bicuspid aortic valve type 1 label, a tricuspid aortic valve label, quadricuspid aortic valve label, pentacuspid aortic valve label, unicuspid aortic valve label, or n-cuspid aortic valve label.

12. The method according to claim 1, wherein at least one of:
the region of interest is related to a left atrial appendage and the classes of the set of classes comprise left atrial appendage morphology labels, such as a cowley flower cauliflower label or a chicken wing label; or
the region of interest is related to a mitral valve of the patient and the classes of the set of classes comprise calcification grade labels, such as wherein the calcification grade labels comprise at least a mild label, a moderate label and a severe label; or
the region of interest is related to an aortic root of the patient and the classes of the set of classes comprise calcification grade labels, such as wherein the calcification grade labels comprise at least a mild label, a moderate label and a severe label; or
the cardiac characteristic relates to a cardiac phase and the classes of the set of classes comprise percentage intervals or percentage labels;
the region of interest is related to a left atrial appendage and the cardiac characteristic relates to a left atrial appendage filling, wherein the classes of the set of classes comprise percentage intervals or percentage labels.

13. A method of training a machine learning data processing model for classifying an image feature in order to determine, from cardiac image data, a cardiac characteristic of a patient by evaluating a probability that the image feature matches each class of a predetermined plurality of classes forming a set of classes such as to characterize the image feature, the method of training, further comprising:
a) receiving, by the machine learning data processing model, a training data set comprising training data, the training data including cardiac image data of at least a part of a cardiac image, and optionally data indicative of a region of interest which includes the image feature;
b) obtaining, by the machine learning data processing model, a plurality of classes forming a set of classes, wherein the classes together describe status classes of the cardiac characteristic;
c) receiving, by the machine learning data processing model, a ground truth data set comprising ground truth data indicative of a ground truth class that matches with the image feature;
d) training the machine learning data processing model based on the training data received in step a), the classes of the set of classes obtained in step b), and the ground truth data received in step c), for enabling the machine learning data processing model, after completion of the training period, to perform the step of providing probability data, the probability data comprising for each class of the set of classes a probability that the image feature matches the respective class.

14. An electronic image processing system, the system comprising a trained machine learning data processing model, for classifying an image feature in order to determine, from cardiac image data, a cardiac characteristic of a patient by evaluating a probability that the image feature matches each class of a predetermined plurality of classes forming a set of classes such as to characterize the image feature, wherein the system further comprises one or more processors and a memory storing the trained machine learning data processing model and one or more instructions, which when loaded into the memory enable the one or more processors to perform the steps of:
receiving, by the trained machine learning data processing model, the cardiac image data;
obtaining, by the trained machine learning data processing model, data indicative of the region of interest;
collecting, from the trained machine learning data processing model, probability data, the probability data comprising for each class of the set of classes a probability that the image feature matches the respective class;
evaluating, by a controller, the probability data for the classes of the set of classes, and identifying one or more candidate classes having a significant probability of matching with the image feature;
determining, by performing a comparison of the probability data for the candidate classes, for each of the identified candidate classes a certainty level indicative of a certainty that the image feature matches the respective candidate class; and
dependent on the step of determining, performing at least one step of:
when the number of candidate classes equals one, and the certainty level exceeds a threshold level: associating the one candidate class with the image feature; or
when the number of candidate classes equals one, and the certainty level is smaller than a threshold level: requesting user input indicative of a confirmation or disaffirmation that the one candidate class matches with the image feature; or
when the number of candidate classes exceeds one: requesting, for each of the one or more candidate classes, user input indicative of a confirmation or disaffirmation that the respective candidate class matches with the image feature.
